(19) United States
(12) Patent Application Publication
(10) Pub. No.: US 2013/0310296 A1
(43) Pub. Date: Nov. 21, 2013

(54) BAR SOAP COMPOSITION WITH ENHANCED SENSORY PROPERTIES IN SOFT AND VERY SOFT WATER

(71) Applicants: Philippe Lizop, Paris (FR); Maud Grasset, Cabrieres d'Avignon (FR); Vineet Mital, Chandivali (IN)

(72) Inventors: Philippe Lizop, Paris (FR); Maud Grasset, Cabrieres d'Avignon (FR); Vineet Mital, Chandivali (IN)

(73) Assignee: SERENA LINLEY SARL, Saint Martin de Castillon (FR)

(21) Appl. No.: 13/139,007

(22) PCT Filed: Apr. 8, 2011
(86) PCT No.: PCT/IN2011/000242

§ 371 Date: Jun. 10, 2011

(30) Foreign Application Priority Data
Feb. 24, 2011 (IN) 510/MUM/2011

(51) Int. Cl. A61K 8/19; A61K 8/92; A61Q 19/10

(52) U.S. Cl. CPC A61K 8/19; A61Q 19/10; A61K 8/92
USPC 510/152

(57) ABSTRACT

The present invention relates to a predominantly soap based bar (e.g. about 50% to 85% by weight soap) specifically a toilet bar used for personal cleansing. In particular it relates to a toilet bar soap which has a specific formulation to take care of sensory attributes (e.g., skin hydration, skin softness, skin after-feel, rinsability & lather) during the washing process in soft and very soft water.

### FIELD OF THE INVENTION

The present invention relates to a predominantly soap based bar (e.g. about 50% to 85% by weight soap) specifically a toilet bar used for personal cleansing. In particular it relates to a toilet bar soap which has a specific formulation to take care of sensory attributes (e.g., skin hydration, skin softness, skin after-feel, rinsability & lather) during the washing process in soft and very soft water.

### BACKGROUND OF THE INVENTION

Soap bars have been known to be the most practiced form of skin wash aids. There is a large & loyal clientele for Bar Soaps. This is because of its economic attractiveness and cleaning efficacy. Cost per wash from a solid soap bar has so far been relatively modest than from equivalent liquid options. Mildness is an issue with all forms of soap formulations. Bars containing predominantly synthetic surfactants are perceived to be milder; but one aspect which many consumers complain about and which synthetic surfactant bars do not provide is the squeaky clean feeling of soap bars. Hence consumers prefer to use soap bars. However despite widespread use, soap bars are not without their share of problems. The performance of the bar (lather, rinsability and skin feel) vary in different types of water, depending upon the characteristics of the water. Consumers notice the difference between the same soap bar performance in hard water and soft water and are dissatisfied with the change in performance with change in the nature of water supply. Specifically in very soft and soft water, rinsability even with soap bars is an issue just like the rinsability profile of synthetic surfactants and therefore consumers complain about this aspect. In prior art, there are no soap bars available which addresses the above problem of the consumers.

Two performance attributes of soap bars, most readily noticed by consumers are lather and rinsability. Soap Bars give high amount of lather in soft water and low amount in hard water. In soft and very soft water soap bar leaves a slippery feeling on the skin which gets removed only on large amount of dilution water. Hence rinsability of Bar Soap in soft and very soft water is a problem when compared to the same bar soap's performance in hard water. This slippery feeling, if not removed by dilution, effectively connotes residual surfactant on the skin which can strip the skin of its essential lipids. This manifests itself in consumers judging soaps to be harsh and therefore look for alternatives.

The composition of the present invention not only solves this problem but also provides good skin hydration with medium spreading & dry feel emollients. Prior art as listed above either deals with mildness of the formulation disregarding the rinsability aspect (very important from the consumer's perspective) or talks about rinsability only in one type of water hardness.

### DETAILED DESCRIPTION OF THE INVENTION

The present invention relates to compositions comprising relatively large amounts of calcium and magnesium salts and Medium Chain Triglycerides. Such compositions are claimed to possess unique sensory benefits in soft and very soft water (Water having hardness from 0- to 70 ppm CaCO3 is defined as very soft water and that having hardness from 70 ppm till 180 ppm CaCO3 is defined as soft water).

Such benefits have not been delivered by any prior art for consumers living with soft and very soft water areas.

The bar composition of the invention comprises about 70 to 95%, preferably 75 to 93% by weight soap. The term “soap” is used herein in its popular sense, i.e., the alkali metal or alkanol ammonium salts of aliphatic, alkane-, or alkene monocarboxylic acids. Sodium, potassium, magnesium, mono-, di- and tri-ethanol ammonium cations, or combinations thereof, are suitable for purposes of this invention. In general, sodium soaps are used in the compositions of this invention, but from about 1% to 5 about 25% of the soap may be potassium or magnesium or calcium soaps. The soaps useful herein are the well-known alkali metal salts of natural of synthetic aliphatic (alkanoic or alkenoic) acids having about 8 to 22 carbon atoms, preferably about 8 to about 18 carbon atoms. They may be described as alkali metal carboxylates of acyclic hydrocarbons having about 8 to about 22 carbon atoms. The soap may be derived from one or mixture of C8-C22 preferably C12-C18 straight or branched chain, saturated and unsaturated monocarboxylic acids of natural or synthetic origin.

Examples thereof include the fatty acids derived from coconut oil, olive oil, palm kernel oil, tall oil, soy bean oil, cottonseed oil, peanut oil, safflower oil, sunflower seed oil, corn oil, fish oils, tallow and the like. For illustration individual fatty acids will include capric, caproic, Laurie, myristic, palmitic, Stearic, oleic, linoleic, linolenic, myristoleic, palmitoleic, behenic, arachidic, ricinoleic and the like. The fatty acids may also be derived synthetically by paraffin oxidation, oxo-synthesis or the like. Generally soaps having fatty acid distribution of either singly or in combination of coconut oil, palm kernel oil, babassu oil, ouricuri oil, tucum oil, cohune nut oil, murumuru oil, khakan kernel oil, dika nut oil may provide lower end of the broad molecular weight range. Those soaps having the fatty acid distribution of tallow, palm, peanut or rapeseed oil, or their hydrogenated derivatives, may provide the upper end of the broad molecular weight ranges.

The squeaky, non-slimy feel or rinsability is desirable in most bar compositions and is often perceived as a sign of cleanliness. This also has implications in terms of actual delivery of mildness in consumer situations rather than the very experimental conditions of mildness predicted in laboratory conditions. Fluorescing dye imaging techniques used in actual consumer wash situations have revealed the extent to which good rinsability can contribute to better mildness. Ref: Soap & Detergent Bar Rinsability: Wortzman et al; J. Soc. Cosmet. Chem., 37, 89-97 (March-April 1986)

Hence, mildness of ingredients alone cannot guarantee good mildness for products unless it is combined with overall product formulation with good rinsability. Alternatively, the manipulation of soap bar formulation to ensure good rinsability can give best mildness achievable in actual usage conditions, to the extent possible while using bars made of predominantly alkali metal soap.

The bar composition of the present invention comprises at least one soluble anionic surfactant and at least one sufficient amount of multivalent cation-containing salt such that the bar composition, during rinsing, passes through an environment where precipitation of surfactant-multivalent cation occurs such that the solution is substantially devoid of micelles.

Surfactants of the present invention are selected from a group consisting of non-ionic surfactants, anionic surfactants, cationic surfactants, amphoteric surfactants, insoluble soap and mixtures thereof.

The surfactant system of the invention comprises two parts: Insoluble and soluble surfactants.

(1) Insoluble surfactant such as alkali metal, long chain (preferably C16 to C24) fatty acid soaps (e.g., calcium/magnesium stearate) and/or insoluble synthetic surfactant; and

(2) Soluble surfactant). Within the group of soluble surfactants (primarily natural or entirely synthetic), some surfactants are more sensitive than others to ion (primarily calcium/magnesium) precipitation.

One of the object of the invention is to formulate the soluble surfactant component so that at least a minimum amount of the soluble surfactant (e.g. 1% of total soluble surfactant up to 100% of total soluble surfactant, preferably 5 to 50%) is sensitive to calcium and other divalent cations (e.g. Ca+2 and Mg+2 found in water).

As mentioned above, insoluble surfactant comprises insoluble fatty acid soap. This includes soaps of C16-C24 fatty acids such as, for example, alkali metal stearate. The insoluble surfactant may also include insoluble synthetic surfactant.

The soluble surfactant may be a soluble soap such as C8-C14 fatty acid soap or any other suitable soluble surfactant sensitive to calcium and magnesium ions. The soluble surfactant in this invention comprises predominantly (at least 75% soluble surfactant) alkali metal soaps of C8 to C14 fatty acids. It may also comprise of synthetic surfactant and such synthetic may be divided into two classes—ion-sensitive soluble synthetic and ion-insensitive soluble synthetic.

The insensitive component is optional (0% to 15% by weight total composition), although it will generally comprise 1% to 10% of the bar composition. Sensitivity or insensitivity is generally measured with regard to calcium ions present in the water during the rinsing process. “Insensitive” means that in water of French Hardness of 6 (60 ppm calcium in water) the surfactant will not precipitate with the calcium and surfactant micelles (believed responsible for “slimy” rather than “squeaky” feel) will remain generally intact.

Alternatively, ion sensitive surfactants, under same conditions, will tend to lose their micellar formation to yield desired “squeaky” feel. Examples of soluble, but insensitive, synthetics include acyl isethionate (e.g., sodium cocoyl isethionate), alkyl sulphate (e.g. sodium dodecyl sulphate), alkyl ether sulfate, alkyl sulfosuccinate (e.g. cocoamidosulfosuccinate), and certain amphoterics (e.g. betaine).

Examples of surfactants sensitive to precipitation include salts of alkyl carboxylate acids (e.g. soaps); carboxylates of formula:

R—(CH2CH2O)nCO2-X

wherein R ranges from C8-C20 alkyl, preferably C12-C18 alkyl, n is from 1 to 40, preferably 2 to 9, and X is sodium, potassium, ammonium or triethanolammonium; carboxylate salts of fatty acyl amino acids (e.g., sodium cocoyl glycinate, sodium lauryol lactylate); monoalkyl phosphates; and certain types of amphoterics (e.g., n-alkyl iminodialkanoate).

As noted, certain categories may overlap; however sensitivity/insensitivity is measured by French Hardness, as mentioned above, and is the determinative factor in deciding what to use.

Multivalent Cations included as part of the composition may be Calcium or Magnesium soluble salts. It is an unexpected and surprising discovery that the multivalent cations have to be present in either discrete domains (thus necessitating their usage in large particles of 0.5-5 mm diameter preferably 1-2 mm dia) or if present in particles lower than 0.5 mm they have to be sufficiently shielded from the water of the formulation so that they are in a substantially intact form in the bar but available for dissolution in the soap-foam-water combination. This can be achieved by coating the particles in a Fluid Bed Dryer/Pan coater with 50-250 micron coatings that are soluble in a large quantity of water but generally water resistant in a bar. Examples of such coatings are polyhydric alcohols of varying Molecular Weight e.g PEG 1500, 4000 or 6000 or hydrophobic coatings which react/disperse in the alkaline soap water medium e.g. Lauric/Myrisitic acids, Beeswax, Shea Butter and the like. Other solid coatings like fumed silica may also be advantageously employed if it fits into the marketability of the final product as a bar with exfoliating properties.

Other key to the formulation is the inclusion of medium spreading and dry feel emollient like Medium Chain Triglycerides (MCTs). Spreadability is measured by the Zeidler Test (Reference Zeidler, U., Über das Spreiten von Lipiden auf der Haut, Fette, Seifen, Anstrichmittel, Vol. 87, No. 10, Diisseldorf, 1985) as described below:

A 4 mg dose of the emollient is applied on the dorsal forearm in an environment of 23° C. and 60% RH. Spreading area calculated after 10 min is tabulated.

According to Zeidler cosmetic emollients can be classified as low spreading (below 300 mm2/10 min), medium spreading (300-1000 mm2/10 min) and high spreading (above 1000 mm2/10 min). This invention uses medium spreading emollients having spreadability of 500-1000 mm2/10 min.

Not wishing to be bound by theory, it is postulated that that the slimy ‘unclean” feeling associated with synthetic surfactants and also with soap bar wash in very soft and soft water conditions is related to charge repulsion. This charge repulsion comes into play due to highly charged surfaces of clean skin as well as due to electrostatic repulsion with charged surfaces of micelles present, either in the surfactant solution present in the interstitial spaces of the skin and/or adsorbed micelles directly on the skin as a function of application of solid soap crystals on to the skin during the washing process. Hence squeaky clean feeling represented by high frictional forces of rubbing can be enhanced for soap bar formulations in soft and very soft water by a formulation which interacts with these charges either by neutralization of charges species and/or dilution of charged species like micelles present and/or by physical barriers using precipitation of multivalent cation sensitive surfactant combination and a medium spreading but dry feeling emollient. Shielding of the Multivalent cation salt is essential so that it does not already interact with the soluble surfactant within the water of the formulation and thus not be available for interaction during the usage of the bar. The choice of emollient is crucial as low spreading emollients will give rise to a greasy feeling and hence the squeaky clean feel will not be achieved while fast spreading emollients will not interact enough with the charged surfaces and therefore not achieve squeaky clean feel.

Composition of the present invention further contains 0% to 5.0%, preferably 0.1% to 3.0% by weight total composition free fatty acid. Generally, at least some free fatty acid is needed to act as pH buffer (keep pH lower) as well as to act as structurant.

Significantly, any free fatty acid present is also long chain, insoluble free fatty acid. For example, the free fatty acid may be C16-C24 free fatty acid. Typically, it may comprise mixtures of C16 and C18 (e.g., palmitic/stearic acid mixtures).

The proportion of stearic acid to Medium Chain Triglyceride is in the range of 3:4 to 1:4.

Typically the bar compositons will further comprise 5-18%, preferably 6-12% by wt. total composition water.

In addition, the bar composition of the present invention may include 0% to 5% by weight optional ingredients as follows:

Fragrances (perfume); sequestering agents such as Tetrasodium ethylenediaminetetraacetic acid salt (EDTA di or tetra sodium salt), Ethane Hydroxy Diphosphonic acid salts or their mixtures in an amount of 0.01 to 1% preferably 0.01 to 0.05% and colouring agents, opacifiers and pearlizers such as zinc stearates, magnesium stearates, Titatnium Dioxides, Ethylene glycol monosterates, reflective mica or coated mica particles; all of which are used to enhance the cosmetic properties or appearance of the product

The composition may also comprise of sodium chloride or sodium sulphate as electrolytes.

Other benefit agents which may be included are mono or polyhydric alcohols like glycerol, sorbitol and polyethylene glycols of various moleculear weights

Other benefit agents which may be included are exfoliants and or appearance enhancers such as polyoxytheylene beads, PMMA beads, oat meal, walnut shells, apricot seeds, lotus flower stems, ground fruit pits, sugar extrudates, mineral agglomerates, loofah bits, beads of starch or maltodextrin and colouring matter or their combinations, cooling agents such as menthol and its various derivatives and lower alcohols, skin calming agents such as aloe vera, essential oils like menthe, jasmine, camphor, bitter orange peel, cedar, cinnamon, bergamot, pine, lavender, bay leaf, clove, hibiscus, eucalyptus, lemon, thyme, rose, sage, eugenol, citral, citronellal, borneol, linalool, etc. Sunscreen agents, antiageing compounds and skin lightening agents may be used if required for the marketing of such soap bars.

Oil control agents including sebum suppressants, mattifiers such as silica, astringents including tannins, plant extracts from green tea and witch-hazel

Antioxidants and chelating agents like Butylated hdroxytoluene and citric acid, or natural antioxidants like rosemary extract may be used if appropriate

Further the bar composition of the invention may include insoluble organic, inorganic or mineral solids as structurants. These include starch (modified or native) or maltodextrins, fumed, precipitated or modified silica, calcium carbonate, kaolin, talc or alumina-silicate clays.

The compositions may further comprise of antimicrobials of synthetic or natural origin like Trichloro carbanilides, 2-hydroxy-4,2′4′ trichlorodiphenylether, zinc salts of ricinoleic acid, tea tree oil etc.

The present invention is set forth in greater details in the examples to follow. The examples are for illustration purposes and are not intended to limit the scope of claims in any way

### EXAMPLES

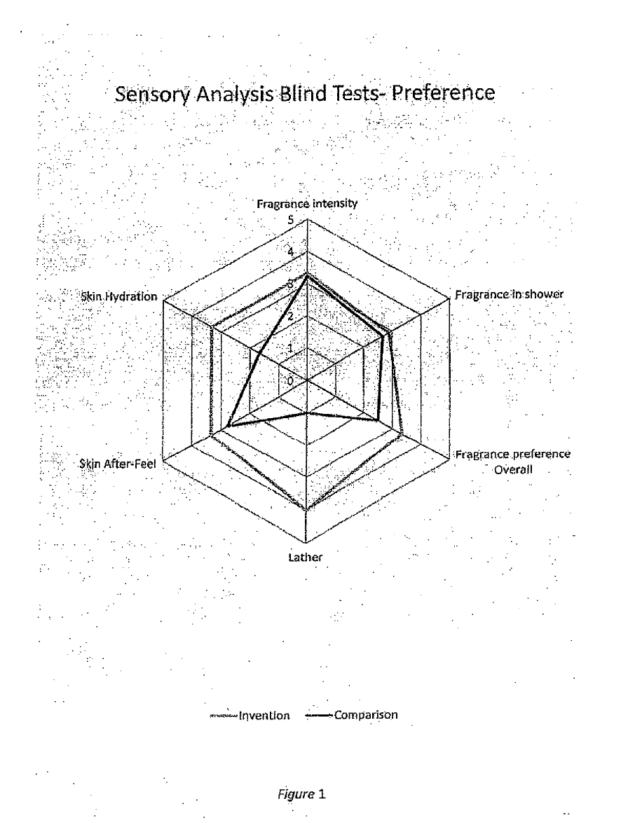

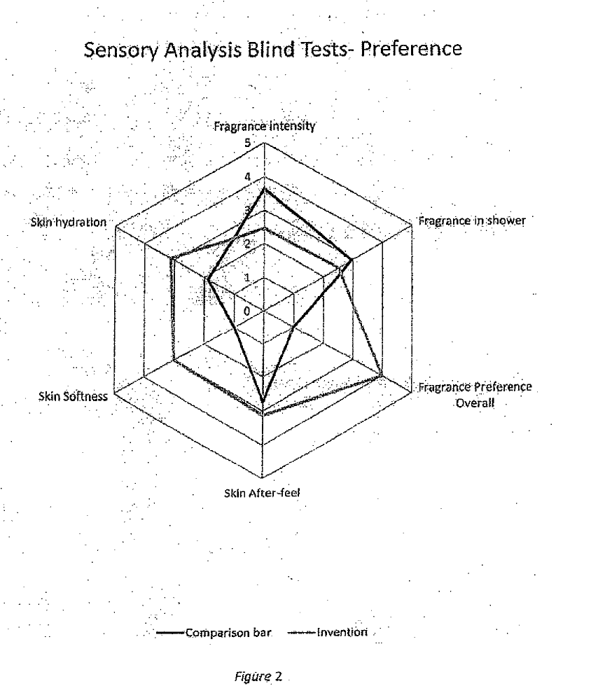

Sensory properties like skin-hydration, skin-feel, lather, skin-softness & overall fragrance properties of the inventive bar soap in comparison with the comparison bar show improved results and are evident in the sensory analysis blind charts mentioned in FIGS. 1 & 2.

### Example 1

| Soap Noodles (80:20 ratio) & Base | to 100 (all in %) |
|---|---|
| Calcium Chloride | 0.2 |
| Magnesium Sulfate | 0.2 |
| Sodium Laureth Sulfate | 0.4 |
| Calcium Stearate | 2.0 |
| Magnesium Stearate | 2.0 |
| Stearic Acid | 1.0 |
| Medium Chain Triglyceride (MCT) | 0.5 |
| Perfume | 1.5 |

### Example 2

Rinsability benefit defined as per the invention demonstrated in the following tabulated data.

| Raw Materials | 1 | 2 | 3 | 4 | 5 | 6 | 7 | 8 | 9 | 10 | 11 | 12 | 13 |
|---|---|---|---|---|---|---|---|---|---|---|---|---|---|
| Soap Base | to 100 | to 100 | to 100 | to 100 | to 100 | to 100 | to 100 | to 100 | to 100 | to 100 | to 100 | to 100 | to 100 |
| Calcium chloride | 0.04 | 0.20 | 0.20 | 0.00 | 0.20 | 0.20 | 0.20 | 0.20 | 0.00 | 0.20 | 2.00 | 0 | 0 |
| dihydrate | | | | | | | | | | | | | |
| Magnesium sulfate | 0.04 | 0.20 | 0.20 | 0.00 | 0.20 | 0.20 | 0.20 | 0.20 | 0.00 | 0.20 | 2.00 | 0 | 0 |
| heptahydrate | | | | | | | | | | | | | |
| Coated Calcium | 0 | 0 | 0 | 0 | 0 | 0 | 0 | 0 | 0 | 0 | 0 | 1 | 0 |
| Chloride Dihydrate | | | | | | | | | | | | | |
| (200 micron Lauric | | | | | | | | | | | | | |
| acid coating) | | | | | | | | | | | | | |
| Coated Magnesium | 0 | 0 | 0 | 0 | 0 | 0 | 0 | 0 | 0 | 0 | 0 | 1 | 0 |
| Sulfate Heptahydrate | | | | | | | | | | | | | |
| (200 micron Lauric | | | | | | | | | | | | | |
| acid coating) | | | | | | | | | | | | | |
| Calcium Chloride | 0 | 0 | 0 | 0 | 0 | 0 | 0 | 0 | 0 | 0 | 0 | 0 | 1.5 |
| Dihydrate 1-2 mm | | | | | | | | | | | | | |
| Magnesium Sulfate | 0 | 0 | 0 | 0 | 0 | 0 | 0 | 0 | 0 | 0 | 0 | 0 | 1.5 |
| Heptahydrate 1-2 mm | | | | | | | | | | | | | |
| Fragrance | 1.30 | 1.30 | 1.30 | 1.30 | 1.30 | 1.30 | 1.30 | 1.30 | 1.30 | 1.50 | 1.50 | 1.5 | 1.5 |
| Sodium Lauryl | | | 0.30 | 0.30 | 0.30 | 0.00 | 0.30 | 0.30 | 0.30 | 0.00 | 0.00 | 0 | 0 |
| sulphate | | | | | | | | | | | | | |
| Calcium stearate | | | | | 2.00 | 0.00 | 0.00 | 2.00 | 2.00 | 2.00 | 0.00 | 2.0 | 2 |
| Magnesium stearate | | | | | 2.00 | 0.00 | 0.00 | 2.00 | 2.00 | 2.00 | 0.00 | 2.0 | 2 |
| Lauryl Alcohol 2EO | | | | | | 2.00 | 0.00 | 0.00 | 0.00 | 0.00 | 0.00 | 0 | 0 |
| Sodium carbonate | | | | | | | 1.00 | 0.00 | 0.00 | 0.00 | 0.00 | 0 | 0 |
| Stearic acid | | | | | | | | 1.00 | 1.00 | 1.00 | 1.00 | 1.0 | 1 |
| Caprylic capric | | | | | | | | 0.50 | 0.50 | 0.50 | 0.50 | 0.5 | 0.5 |
| triglyceride | | | | | | | | | | | | | |
| Guar Hydroxypropyl | | | | | | | | | 1.00 | 0.00 | 0.00 | 0 | 0 |
| Trimonium Chloride | | | | | | | | | | | | | |
| Sodium Laureth | | | | | | | | | | 0.40 | 0.40 | 0.4 | 0.4 |
| Sulfate | | | | | | | | | | | | | |

Rinsability

| | | | | | | | | | | | | | |
|---|---|---|---|---|---|---|---|---|---|---|---|---|---|
| demineraiised water | VS | VS | VS | VS | VS | VS | VS | F | S | F | F | OK | OK |
| soft water 50 ppm | VS | VS | VS | VS | VS | VS | VS | F | S | F | F | OK | OK |
| soft water 75 ppm | S | S | S | S | S | VS | S | OK | S | OK | OK | OK | OK |
| soft water 100 ppm | S | S | S | S | S | S | S | OK | S | OK | OK | Ok | Ok |
| soft water 180 ppm | OK | OK | OK | OK | OK | OK | OK | OK | Ok | OK | OK | D | D |

| | | |
|---|---|---|
| where | | |
| Description | Score | Code |
| Very Slippery | 5 | VS |
| Slippery | 4 | S |
| Fair | 3 | F |
| Squeaky Clean | 2 | OK |
| Draggy | 1 | D |

We claim:

1. A soap bar composition comprising:
i. 1.0% to 5.0% of its weight formed by a combination comprising:
a) at least one water-soluble anionic surfactant forming 6.0 to 12.0% of the weight of the combination, the weight of the at least one water-soluble anionic surfactant being less than 1% of the weight of the soap bar composition and the at least one water-soluble anionic surfactant being sensitive to ion precipitation by cations in water having French Hardness of 2.5 to 18.0; and
b) such quantity of at least one multivalent cation-containing salt as to substantially deplete the soap-bar composition of surfactant micelle by forming surfactant-multivalent salt precipitate;
ii. 0.5% to 5.0% of its weight formed by medium spreading & dry feel emollients having spreadability in the range of 500 to 1000 mm²/10 min (Zeidler test);
iii. 1.5% of its weight formed by perfume; and
iv. the remaining percentage of its weight formed by soap base comprising of predominantly alkali metal salts of fatty acids, water, electrolytes, opacifiers, sequestering or chelating agents and other benefit agents

2. The composition according to claim 1 wherein the at least one water-soluble anionic surfactant comprises alkali metal C₈ to C₁₄ fatty acid soap.

3. The composition according to claim 1 wherein the medium spreading & dry feel emollients comprise stearic acid and Medium Chain Triglyceride.

4. The composition according to claim 3 comprising stearic acid and Medium Chain Triglyceride, wherein the ratio of stearic acid to Medium Chain Triglyceride is 3:4 to 1:4.

5. The composition according to claim 1 wherein the cation of the at least one multivalent cation-containing salt is calcium. This salt is present as discrete particles of 0.5-5 mm or coated particles of size lesser than 0.5 mm

6. The composition according to claim 1 wherein the cation of the at least one multivalent cation-containing salt is Magnesium. This salt is present as discrete particles of 0.5-5 mm or coated particles of size lesser than 0.5 mm

7. The composition according to claim 1 resulting in enhanced squeaky, non-slimy rinse sensation when used for washing with predominantly soap bars in very soft and soft water
